(19) United States
(12) Patent Application Publication
(10) Pub. No.: US 2021/0298953 A1
(43) Pub. Date: Sep. 30, 2021

(54) MINIATURE PRECISION MEDICAL DEVICE

(71) Applicant: SCOUTCAM LTD., Omer (IL)

(72) Inventors: Amir GOVRIN, Ramat Gan (IL); Yekaterina DLUGACH, Mabuim (IL); Richard FOULKES, Lombard, IL (US)

(21) Appl. No.: 17/217,271

(22) Filed: Mar. 30, 2021

Related U.S. Application Data
(60) Provisional application No. 63/001,583, filed on Mar. 30, 2020.

(51) Int. Cl. A61F 9/007; A61B 1/05; A61B 1/018; A61B 1/06; A61B 1/07; A61B 1/00

(52) U.S. Cl. CPC A61F 9/00736; A61B 1/05; A61B 1/018; A61B 1/0684; A61B 2217/007; A61B 1/0638; A61B 1/00009; A61B 1/00016; A61B 2217/005; A61B 1/07

(57) ABSTRACT

A miniature precision medical device comprising an endoscope with at least one camera, wherein at least one sensor of the at least one camera is distally located at a tip of a shaft of the endoscope, characterized with that one or more unaccommodated areas within the shaft, surrounding or next to the at least one sensor, is utilized for accommodating at least one accessory such as illumination source, irrigation tool, suctioning tool, etc.

### FIELD OF THE INVENTION

The invention relates to a miniature precision medical device provided with a high-resolution optic visioning means (e.g., of at least 300 pixels per inch), which can be used in microsurgery or diagnostics such as ophthalmology, neurosurgery, plastic surgery, otorhinolaryngology, Ear-Nose-Throat, and/or other medical fields in which the penetration is limited due the healing process (e.g., inner eye, eardrum—the penetration should be done by small-gauge needle), in laparoscopic procedures, endoscopic procedures, catheterization (vascular, bronchoscopy, nephrology, cardio, biopsy, etc.) and/or other human and/or animal surgical fields.

### BACKGROUND OF THE INVENTION

Miniaturization can be a key to successfully performing precision surgery (i.e., to a high resolution), especially in an intricate organ such as the eye.

It is an object of the present invention to miniaturize several components within one tool for microsurgery procedures. Furthermore, such miniaturization is key to successfully performing precision surgery (i.e., to a high resolution), especially in an intricate organ such as the eye. One or more of the following features can be incorporated in to this tool: a camera, light source, laser source, scraping tool, irrigation (inflow and outflow), aspiration and ultrasound. By incorporating several of these features in to one tool, the procedure is made less cumbersome by not necessitating the use of an excessive surgical toolbox.

It is another object of the present invention to combine optic visioning means with “power washing” of cells and viscoelastic from the lens capsule after cataract removal with the ability to distend the capsule to improve the efficacy and safety of removal of further dislodged cells.

It is yet another object of the present invention to ensure that changes in pressure within the eye are minimal during the procedure.

It is still another object of the present invention to use controlled and targeted jetting of saline in eye surgery to minimize the surgical damage.

It is still another object of the present invention to provide visualization of ciliary sulcus and sub iris area to assist micro invasive glaucoma surgery. Camera device potentially can decline the need of using the goniometer device. Combined optic vision to allow implants (stent), shunt or any other device reduces the eye pressure to be delivered and placed or to allow delivering the shunt.

Tool camera and small channel. Drug accurately can be delivered under direct vision.

Laser and camera: deliver laser power under direct vision to use in glaucoma, to repair tear in retina, to cauterize blood vessels.

It is a further object of the present invention to enable procedures to be standardized by controlling flow-rate (and hence force), which will inevitably improve the opportunities for successful implementation of procedures in eye surgery.

It is a further object of the present invention to enable cataract surgery, glaucoma surgery and vitrectomy, tear duct opening, eye trauma, and tumour surgery.

### SUMMARY OF THE INVENTION

A miniature precision medical device comprising an endoscope with at least one camera, wherein at least one sensor of the at least one camera is distally located at a tip of a shaft of said endoscope, characterized with that one or more unaccommodated areas within said shaft, next to or surrounding said at least one sensor is utilized for accommodating at least one accessory.

In one aspect, the at least one camera is adapted to provide high-resolution images of at least 30K pixels.

In another aspect, the tip is configured to be inserted through an incision made around the cornea during cataract surgery and minimal invasive glaucoma surgeries (MIGS).

In another aspect, the tip is configured to be inserted through the trocars used in vitrectomy surgery.

In yet another aspect, the diameter of said device is adapted to enable minimally invasive surgeries that employ surgical techniques meant to limit the size of incisions needed of less than 3 mm and so lessen wound healing time, associated pain, or risk of infection. For example, the diameter of the tip is less than 1.8 mm.

According to an embodiment of the invention, one or more illumination sources are incorporated in to said device via at least one of the unaccommodated areas. In one aspect, the illumination source is located distally at the tip or proximally in a hand-piece of the device, or at the connector, or at a video controller. The distal illumination source can be one or more LEDs. Alternatively, the light may reach the distal end of the endoscope via optic fibers and/or light guides.

In yet another aspect, a phaco-emulsifier tool is integrated in to said device. For example, the phaco-emulsifier tool can be circular or elliptical.

According to some embodiments, the device further comprises a tool for removing particles of lenses under direct vision. For example, the tool for removing particles of lenses under direct vision is an irrigation tool, an aspiration tool, a camera, or a combination thereof. According to another embodiment, the irrigation and aspiration are integrated together in concentric circles, wherein one in the inner circle and one in the outer circle, or they are located adjacently.

In yet another aspect, the irrigation tool having an irrigation port that is shaped to control pressure by fluidics passing through the tip.

In still another aspect, the camera is integrated in to an olive tip irrigation cannula for allowing scarping the lens of the eye.

According to an embodiment of the invention, the camera is attached to a cannula.

According to yet another embodiment, a laser device is incorporated in to said multi-purpose eye surgery device via at least one of the unaccommodated areas.

According to yet another embodiment, an external illumination source is adapted to provide illumination in accordance with the orientation of the device/camera.

According to yet another embodiment, a tube or the tip of the endoscope is made of a transparent material (e.g., a polymer), thus it can be used as light guiding generated by an illumination source.

According to yet another embodiment, the wavelength of the light can be varied.

In another aspect, the device comprises an imager for converting an optical image viewed by the endoscope into digital image data, a processor for processing the digital image data, and a communication module configured for transmitting the processed digital image data to a remote receiver in a wireless or wired manner.

According to an embodiment of the invention, the communication module comprises one or more antennas for directing wireless signals relative to said digital image data to the remote receiver.

In still another aspect, the device further comprises a hand-piece that includes a body cavity or an insertion socket through which the endoscope is detachably attachable to the device.

According to an embodiment of the invention, the processer is part of a control module that is detachably attachable to the device.

According to an embodiment of the invention, the tip is detachably attachable to a hand-piece of the device.

According to an embodiment of the invention, the device further comprises a hand-piece that has a straight tip designed for the back of the eye surgeries.

According to an embodiment of the invention, the device further comprises a hand-piece that has a curved tip designed for the front of the eye surgeries.

According to an embodiment of the invention, a hand-piece of said device has a bendable and articulated tip.

According to an embodiment of the invention, the device is disposable.

### BRIEF DESCRIPTION OF THE DRAWINGS

In the drawings:

FIG. 1 schematically illustrates a section view of a tip of an endoscope's shaft having an essentially circular form, according to an embodiment of the invention;

FIG. 2 schematically illustrates a section view of a tip of an endoscope's shaft having an essentially elliptical form, according to an embodiment of the invention;

FIG. 3A schematically illustrates an eye-surgery device with an illumination source (e.g., a light source like LED, a xenon arc lamp, etc.), according to an embodiment of the invention;

FIG. 3B schematically illustrates an eye-surgery device with illumination source like LED, xenon arc lamp etc. located on the distal hand-piece, according to an embodiment of the invention;

FIG. 3C schematically illustrates an eye-surgery device with optic fibres and illumination source located at a proximal end of hand-piece, according to an embodiment of the invention;

FIG. 4A schematically illustrates a side view of the tip of an eye-surgery device with camera and LEDs, according to an embodiment of the invention;

FIG. 4B schematically illustrates a cross-section view of the tip of FIG. 4A;

FIG. 5 schematically illustrates the tip of an eye-surgery device with camera and LED on the same PCB in one embodiment, according to an embodiment of the invention;

FIG. 6 schematically illustrates a side view of the tip of an eye-surgery device with camera and LED behind it, according to an embodiment of the invention;

FIG. 7 schematically illustrates a cross section of the eye-surgery device provided with laser, camera and illumination, according to an embodiment of the invention;

FIG. 8 schematically illustrates an olive type tip of the eye-surgery device, according to an embodiment of the invention;

FIG. 9 schematically illustrates bi-manual aspiration-irrigation tool for the eye-surgery device, according to an embodiment of the invention;

FIG. 10 schematically illustrates a disposable tip with reusable hand-piece, according to an embodiment of the invention;

FIG. 11 schematically illustrates an irrigation tube cross section, according to an embodiment of the invention;

FIG. 12 schematically illustrates the distal tip of the eye-surgery device provided with an opening for irrigation or vacuum, according to an embodiment of the invention;

FIG. 13 schematically illustrates a cross section of the distal tip showing openings for irrigation or vacuum, according to an embodiment of the invention;

FIG. 14 schematically illustrate an eye-surgery device provided with irrigation and a camera sensor located at the tip of the device, according to another embodiment of the invention;

FIG. 15A-15B schematically illustrates an eye-surgery device with a plurality of LEDs and illumination source located at a proximal end of hand-piece, according to an embodiment of the invention;

FIG. 17 schematically illustrates combined camera and irrigation probe working together in parallel with a light probe within a human eye, according to an embodiment of the invention;

FIG. 18 shows the miniaturization capability of a medical ophthalmic device in a medical system, according to an embodiment of the invention; and

FIG. 19 schematically illustrates a block diagram of the electrical components of a miniature precision medical device, according to an embodiment of the invention.

### DETAILED DESCRIPTION OF THE INVENTION

The present invention is a miniature precision medical device comprising an endoscope with at least one camera (e.g., a video camera), wherein the sensor of the at least one camera is distally located at the tip of the endoscope's shaft (e.g., to be inserted into the eye for imaging from within).

While the endoscope's shaft is minimized for enabling its insertion through small surgery incision, the proposed device is designed to optimally utilize the inner section area of the shaft's tip, by utilizing unaccommodated areas surrounding the camera's sensor. These unaccommodated areas can be used to receive one or more accessories, such as illumination sources (LEDs, fibre-optic, etc.), laser fiber, irrigation tool, suctioning tool, phaco-emulsifier tool, a tool for removing particles of lenses under direct vision, or any other accessory suitable to be delivered/installed within one or more of these unaccommodated areas.

Throughout this description the term “shaft” is used to indicate a tube-like structure adapted to accommodate at least a camera therein. This term does not imply any particular shape, construction material or geometry, and invention is applicable to all suitable structures. For example, the tube-like structure can be hollow, multichannel, built from polymer, metal, or combination thereof, can be integral, or build from different elements, can be flexible, rigid or semi flexible, semi rigid, etc.

According to an embodiment of the invention, the miniaturization enables to obtain high quality images (e.g., providing quality of at least 30k pixels in relation to image sensors of the camera), while the body of the device is small enough to be inserted through an incision made around the cornea (e.g., during cataract surgery and MIGS-minimal invasive glaucoma surgeries). According to some embodiments of the invention, the diameter of the device is less than 1.8 mm, thereby enabling to perform minimally invasive surgeries (i.e., enabling surgical techniques that limit the size of incisions needed and so lessen wound healing time, associated pain and risk of infection). In other words, the present invention relates to a surgery device provided with an optic visioning means in a way that enables to miniaturize several components within one tool for microsurgery procedures.

Reference will now be made to several embodiments of the present invention, examples of which are illustrated in the accompanying figures. Herein several embodiments of the present invention are described for purposes of illustration only. One skilled in the art will readily recognize from the following description that alternative embodiments of the structures illustrated herein may be employed without departing from the principles of the invention described herein.

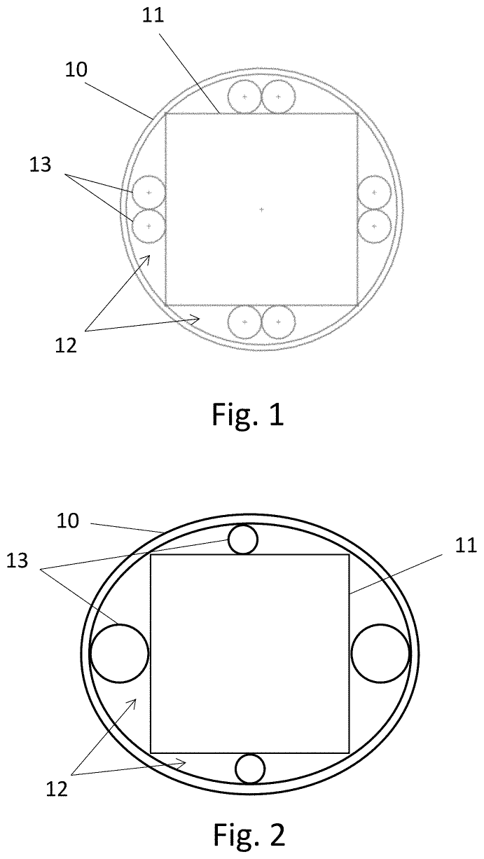

FIGS. 1-2 schematically illustrate a section view of the tip of an endoscope's shaft, according to embodiments of the invention. The square sectional camera sensor 11 inscribed within the round sectional shaft 10 leaves four unaccommodated areas 12 surrounding sensor 11, which are utilized for adding further illumination and surgery tools 13 as will be further described in the following figures. For example, the size of the square sectional camera sensor 11 is about 0.6 mm×0.6 mm, thus the diameter of the round sectional shaft 10 is about 1.5-1.8 mm). The cross-section form of the shaft may vary from one application to another. For example, in FIG. 1, the round sectional shaft 10 is having an essentially circular form, while in FIG. 2, the round sectional shaft 10 is having an essentially elliptical form. Of course, unaccommodated areas 12 of other suitable sectional shapes of shaft can also be used.

According to an embodiment of the invention, the optic visioning means is a camera (e.g., a video camera) incorporated into the surgery device. An optical fibre attachment to these tools may broaden the scope of the utility, efficacy and functionality of the tool. In some embodiments, fibre optic or LED may deliver light of varying colour and intensity to visualize the lens cells for removal.

In another embodiment of the invention, a light source can be placed: i) distally on the tool i.e., direct illumination, or ii) within the body of the tip including light-guides to the distal end, or iii) on the outer body of the tool with optical fibres guiding the light on to the target area. Cells illuminated under different light wavelength will be stained differently and thus illumination is crucial for identification purposes.

In another embodiment of the invention, a laser is incorporated in to the tool to perform a variety of optical procedures e.g. measuring distance, laser cutting, photo-therapy, laser eye surgery, photo-ablation, etc. Furthermore, the wavelength of the laser light can be varied for the specific requirements of the task.

In another embodiment of the invention, an ultrasound tool is incorporated in to the device for destroying lens to small pieces.

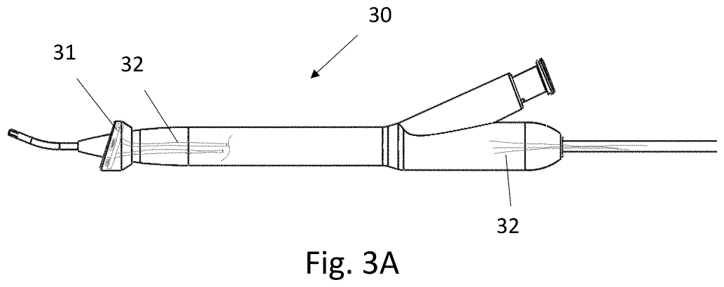

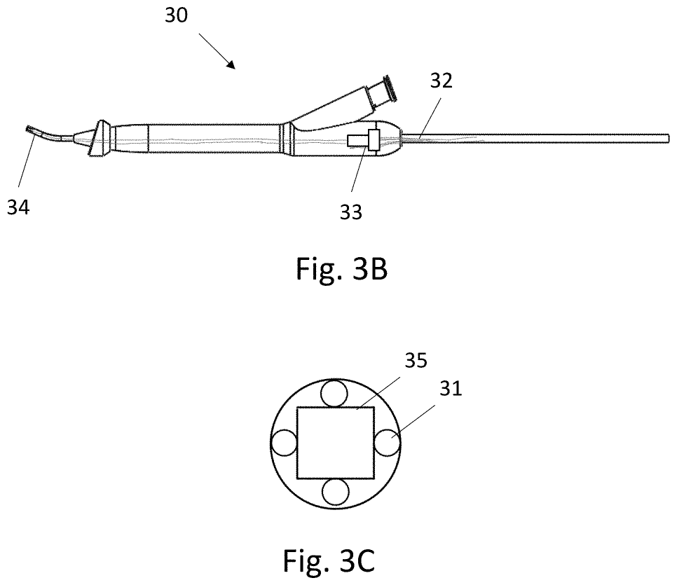

According to an embodiment of the invention, the visualization probe comprises a camera that is incorporated into the medical ophthalmic device. An optical fibre attachment to these tools may broaden the scope of the utility, efficacy and functionality of the tool. In some embodiments, fibre optic or LED can be used to deliver light of varying colour and intensity to visualize the lens cells for removal, e.g., as shown with respect to FIGS. 3A-3C.

FIG. 3A schematically illustrates a medical ophthalmic device 30 with an illumination source 31 (e.g., like a plurality of LEDs, xenon arc lamp etc.) located adjacent to the distal end of device 30, according to an embodiment of the invention. For example, in case the illumination source 31 are one or more LEDs, and numeral 32 indicates the wiring from a power source or an illumination control module connected at the proximal end of device 30. FIG. 3B schematically illustrates the medical ophthalmic device 30 with illumination source that comprises LEDs and fiber-optics, according to an embodiment of the invention. In this embodiment of FIG. 3B, device 30 comprises an element 33 for coupling between LEDs and optic fibres 34.

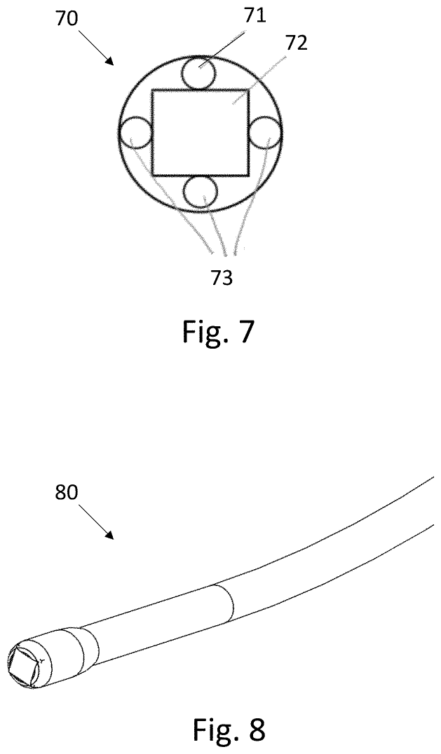

FIG. 3C schematically illustrates a cross-section view of the tip portion of the medical ophthalmic device of FIG. 3A, in which an arrangement of 4 LEDs 31 that surround a sensor 35 of a camera, according to an embodiment of the invention. FIG. 7 schematically illustrates a cross section view of a tip 70 of a medical ophthalmic device provided with a laser fiber 71, a camera 72 and an illumination source 73, according to an embodiment of the invention. FIG. 8 schematically illustrates an olive type tip 80 of a medical ophthalmic device, according to an embodiment of the invention.

Since the light source can be external, the tool is compatible with any externally connected light source. For example see FIG. 3B and FIG. 3C. The ability to see the cells at the angle of the tip of the instrument allows the surgeon to complete the task of cell removal in areas of the eye that are not visible currently from the microscope above. The cross illumination of LEDs and the chromatic fiber optic combined with video observation are key to the performance of the tasks that the tool is capable of. The angle and placement of the video camera is not limited. Typically the video camera may be 1.0 mm size (or even less than 1.0 mm) and incorporate the vacuum part of the bimodal system. This unit may have a slit opening for vacuum of 0.2 mm or greater and may be 1.5 mm wide. FIGS. 3-8 show a variety of options available for the positioning of the camera and light source i.e., the light source can be external and guided by light guides and/or optical fibers, or integrated distally on the tip itself (i.e., internally).

The light source itself can include, but is not limited to: a microscope light, incandescent bulb, operating room light, light emitting diode (LED), fluorescent bulbs, xenon arc lamp, and the like; which can be used individually or in combination. According to an embodiment of the invention, the light source is in the form of a light probe that is adapted to be inserted in parallel to the device. Having light sources of different types available is important because surgical procedures often require procedure-specific lighting. Furthermore, light illumination can be facilitated by light guides and optic fibres (see FIG. 3A and FIG. 3C). In addition, the colour of the light source is not limited to white light alone, but can include any colour and or wavelength that assists the user in carrying out the procedure. According to an embodiment of the invention, a removable distal tip may include one or more LEDs with different wavelength. For example, the colour can be managed by software when RGB LED used.

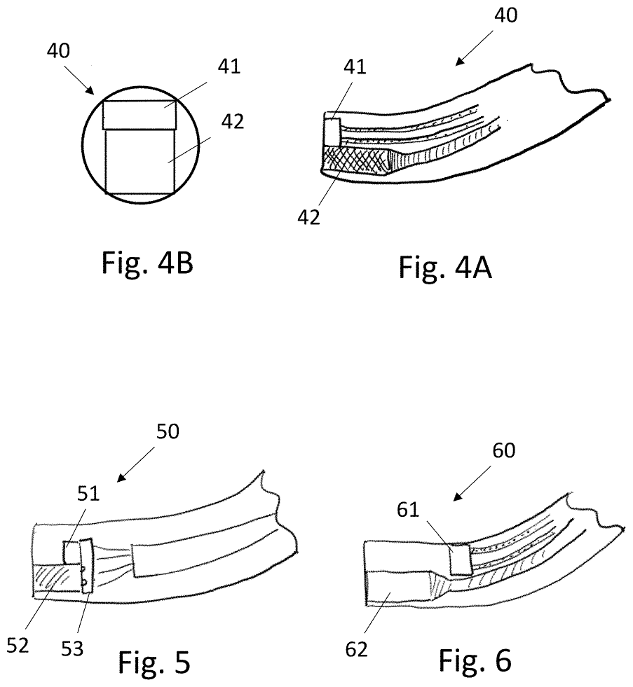

Since the light source can be external, the device is compatible with any externally connected light source. For example, the ability to see the cells at the angle of the tip of the instrument allows the surgeon to complete the task of cell removal in areas of the eye that are not visible currently from existing suctions, such as a microscope above the eye of a patient. The cross illumination of LEDs and the chromatic fiber-optic combined with video observation are key to the performance of the tasks that the device is capable of. The angle and placement of the video camera is not limited. Typically, the video camera may be 1.0 mm size (or even less than 1.0 mm) and incorporate the vacuum part of the bimodal system or the ejection component or both. This unit may have a slit opening for vacuum of 0.2 mm or greater and may be 1.8 mm wide. FIGS. 3A-8 show a variety of options available for the positioning of the camera and light source i.e., the light source can be external and guided by light guides and/or optical fibers, or integrated distally on the tip itself (i.e., internally). For example, FIGS. 4A and 4B show an arrangement in which an illumination source 41 and a camera 42 are located at a tip 40 of a medical ophthalmic device, according to an embodiment of the invention. In this arraignment, illumination source 41 located on top of camera 42. FIG. 5 schematically illustrates a side view of a tip 50 of a medical ophthalmic device with camera 52 and LED 51 on the same PCB 53 in one embodiment, according to an embodiment of the invention. FIG. 6 schematically illustrates a side view of a tip 60 of a medical ophthalmic device with a camera 61 and LED 62 behind it, according to an embodiment of the invention.

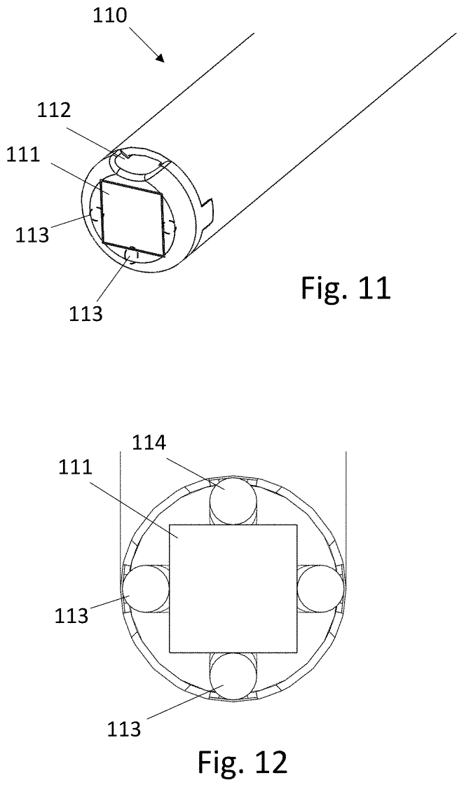

In one embodiment of the invention, the flowrate of fluid through the tool can be controlled by the width and angle of said tool. Furthermore, the water-flow can be pulsed or continuous, according to the surgeon's requirements. The flow may be directed by a pump that is either from the cataract removal unit or the vitrectomy unit. It may also have its own source. The shape and cross section of the exit port of the fluid flow tips can be of varied. If the effluent is too rapid, or its cross-section is too narrow, it may tear through the capsule. Furthermore, the pulsation rate and flow per pulsation has a dynamic effect in relation to the effluent width and height as well as the velocity of the effluent. If the pulsation rate is fast the shock wave of effluent can tear through the capsule. The limits of these energies will be set into the software of the pump to prevent the surgeon from exceeding these limits. See FIGS. 11-13 for examples of embodiments of the invention wherein fluid passes between the optic fibers and/or the camera which is arranged at the distal tip and the openings define the flow direction and pressure.

Furthermore, the pulsation rate and flow per pulsation has a dynamic effect in relation to the effluent width and height as well as the velocity of the effluent. If the pulsation rate is fast the shock wave of effluent can tear through the capsule. See FIGS. 11-14 for examples of embodiments of the invention wherein fluid passes between the optic fibers and/or the camera which is arranged at the distal tip and the openings define the flow direction and pressure.

In another embodiment of the invention, a soft scraping tool of any soft flexible material (e.g. silicone) that is curved at the base and side can be incorporated in to the device. The soft scraping tool can be used to loosen cells that are strongly adhered to the capsule and which can be subsequently swept away with a reverse painting motion of the scraping tool. The device may include a curved or straight section behind the tip to allow for gentle motion against the taught capsule for mechanically removing cells or loosening cells to be swept clear by the tip.

In another embodiment of the invention, the tip may be angled to allow for sub capsular angulation as needed and may be sleeved to allow for inflow to or from an outer sleeve. The fluidics passing through the tip flows at rate that is proportional to resistance/flow. If the flow remains constant, the resistance increases the pressure which causes the fluid to eject at a faster rate. In general an object of this invention is to create a flat or slightly curved fluid plane that will encounter resistance as it flows against the lenticular capsule. The resistance will be adherent cells and viscoelastic that is remaining in the aqueous fluid of the anterior and posterior chambers. Therefore the shape of the tip is essential to the concept of this tool.

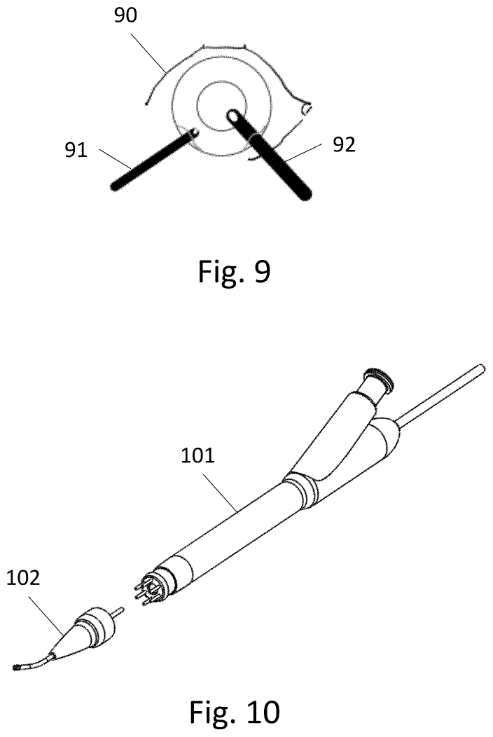

In another embodiment of the invention, the ejection portion of the device may be separated from the suction or return portion in a bi-manual version. In this way the ejection portion size may be reduced to allow it to be placed through a smaller incision as in the side port or an additional incision. See FIG. 9 for a depiction of this embodiment, in which bi-manual mode is used with a camera and irrigation tool 91 are inserted into an eye 90 via a secondary incision, while an aspiration tool 92 is being inserted in eye 90 via a primary incision.

In another embodiment of the invention, the suction portion of the unit may incorporate the light source, the video camera or the injection unit for placing staining compounds onto the capsule. The staining material could be used to visualize lens cells as well as residual viscoelastic. The use of chromatic light on the device will enhance visibility of residual cells. As this tool places visibility behind the iris, it may be equipped with an attachment for placing grasping or cutting tools for performing procedures out of sight from the microscope above.

In another embodiment the camera is integrated into the tool which uses irrigation and aspiration. The Camera and illumination may be integrated into the irrigation tool only (bimanual method for cataract surgery). For the purposes of example alone, the irrigation channel may be at least 0.3 mm with any cross section shape (e.g. a few small tubes or in the free space between the components in the outer tube). The outer diameter can maximally be 1.4 mm. The tool could also be inserted through the primary and/or secondary incision. According to the convenience of the surgical practice, the tips can be either permanent (reusable) or detachable (disposable). See FIG. 10 for a depiction of a disposable tip with hand-piece.

In all embodiments the camera may be integrated in to an olive tip irrigation cannula or any other scarping element.

In another embodiment the camera is integrated into the tool which uses irrigation and aspiration. The camera and illumination may be integrated into the irrigation tool only (bimanual method for cataract surgery). For the purposes of example alone, the irrigation channel may be at least 0.3 mm with any cross-section shape (e.g. a few small tubes or in the free space between the components in the outer tube). For example, the outer diameter can be about 1.5 mm. The tool could also be inserted through the primary and/or secondary incision. According to the convenience of the surgical practice, the tips can be either permanent (reusable) or detachable (disposable). See FIG. 10 for a depiction of a disposable tip 102 with a reusable handpiece 101.

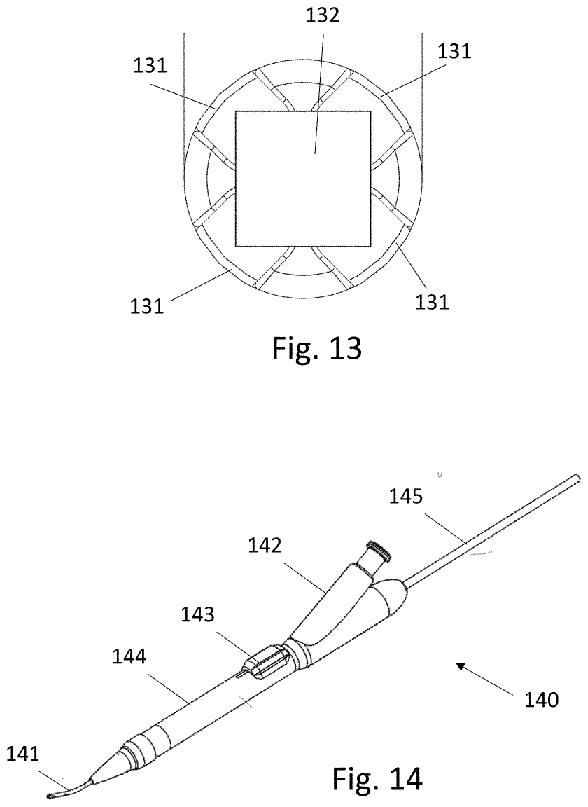

FIG. 11 schematically illustrates a medical ophthalmic device 110 provided with an opening 112 for irrigation or vacuum, according to an embodiment of the invention. Medical ophthalmic device 110 comprises a camera 111, an opening 112 for irrigation or vacuum and illumination sources 113. FIG. 12 schematically illustrates a cross-section of the tip of medical ophthalmic device 110 that shows an irrigation tube 114, according to an embodiment of the invention. In this embodiment, the water flow through irrigation tube 114 and ejected via opening 112. FIG. 13 schematically illustrates a cross-section of a distal tip of a medical ophthalmic device showing openings 131 for irrigation or vacuum that are arranged around a camera 132 located at the center of the tip, according to another embodiment of the invention.

FIG. 14 schematically illustrates a medical ophthalmic device 140, according to another embodiment of the invention. Device 140 comprises an irrigation port 142, a tip 141 on which a camera sensor (not shown) is located. The flow rate can be controlled via a flow rate controller 143 located on the body 144 of device's handpiece. In this embodiment, device 140 is controlled and powered via an operation unit (not shown) that is electrically connected to device 140 via cables 145.

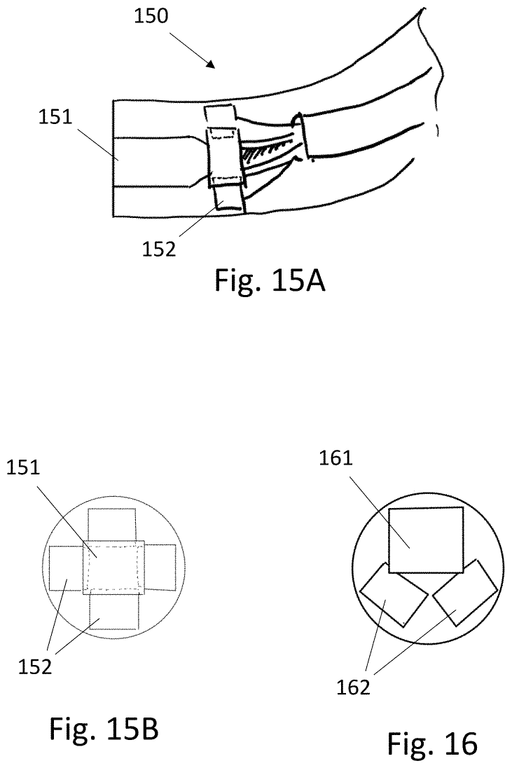

FIGS. 15A-15B schematically illustrate a medical ophthalmic device 150 with plurality of LEDs 152 and a camera 151 located at proximal end of handpiece, according to an embodiment of the invention. FIG. 16 schematically illustrates a cross-section view of another arrangement of a camera 161 and a plurality of LEDs 162 located at proximal end of handpiece.

In another embodiment of the invention the tip may be angled to express the sheet of fluid in a downward or upward direction. It can be angled against the surface of the membrane putting minimal tension on the capsule while “peeling” off the debris and lenticular cells that remain adherent. Therefore the tip gap and width are very important to the function and safety of the device. The width and gap of the tip will have several variations and the user will be able to select the tip that best suits their experience and goal. For the purposes of example alone, the tip can be 0.5 mm to 2.5 mm in width or greater. For the purposes of example alone, the gap can vary from 0.05 mm to 0.5 mm and may include a curved or straight shape. The tip may also have a roughened under-surface to allow for loosening of the cells from the surface behind the jet of fluid. In this way the capsule may be flattened in front of the roughened area to reduce the risk of the capsule rolling or folding and then snagging and tearing. The roughened area may also be widened to allow for a widened surface area to be cleared or loosened ahead of the hydro-dissection cannulated tip that is following. In this way the risk of stroking the delicate capsule is decreased.

In another embodiment of the present invention the material of the tip may be metal with a combination of soft polymer elements to allow gentle scarping.

Furthermore, the tip may have adjustable stiffness enabled by piezo elements, removable metal wire inside, using two tubes, outer rigid tubes and a pre-shaped semi-rigid or flexible inner tube.

The present invention enables procedures wherein lens fragments are removed under direct vision. This can be done with the following combination of components, but is not limited to this list alone: a camera with irrigation, a camera with aspiration, a camera with irrigation and aspiration, a camera and phacoemulsification ultrasound probe, a camera and feature allowing length accurate rotating and positions.

The present invention enables glaucoma surgery. A camera can remove the need for using a goniometer since it can provide visualization of ciliary sulcus and sub iris space. In this use the camera can be attached to a therapeutic device and also to be the diagnostic observational device inserted through the second incision. The camera can also be placed in parallel with a therapeutic device such as a stent delivery tool, shunt delivery tool or the shunt itself. According to some embodiments of the invention, the camera can be integrated to implant delivery tool.

The present invention enables vitrectomy procedures wherein a camera inserted into the eye can serve as a visualization tool when an anterior segment of the eye is opaque (e.g. an opaque cornea). In this embodiment, the camera is small enough to pass through the trocar used in surgery and can assist in positioning sutures, removal of intraocular foreign bodies. For anterior procedures curved tips can be used whereas to vitreoretinal procedures straight tips can be used.

A curved band which may be made of flexible material like silicone can be placed behind the ejection zone. This can be used to stroke the tissue that is being flattened out in front of the effluent. The curved zone behind the tip may be a hard material based on the surgeon's preference for the required task.

The curved tip may mimic the posterior curve of the capsule. The ideal angulation of the tip for hydro-dissection may be effectively parallel or (for the purposes of example alone) may also be angled at 5 to 25 degree or more. This may be accomplished by angling the external instrument or may be built into the tip with the scarping element whether (e.g. silicone) or rough (e.g. diamond) or a rough synthetic or metal surface.

The water inflow is matched by outflow through the sides of the device or by a “bi-manual” technique. In bi-manual mode the tool both irrigates and aspirates via adjacent channels on the same tool i.e., without the need for two separate tools. The tip may also be used as a tool to work against a distended capsule which is less at risk of snagging and tearing. The viscoelastic can be fully ejected from behind and in front of the IOL as well as into the iris sulcus and the dome of the cornea.

The tip may be used for sub-incision clearance as well by directing the flow from the side port or secondary incision. The more completely the eye is cleared of cells the fewer the repercussions expected at the recovery stage.

Lens particles, cortical and viscoelastic material are often concealed underneath the iris. Reaching these with a suctioning tool may damage the capsule resulting in zonular dehiscence or capsular rupture. The superior advantage of power washing versus suction is safety and effectivity. Staining and illumination allow visualization and confirmation of removal.

In one embodiment of the invention, the device can be equipped with an attachment beyond the camera like a forceps or scissors and would allow the surgeon to perform tasks under direct visualization. This could include suturing in a capsular tension ring or a secondary IOL and for releasing an IOL from its capsular attachment. The water ejection system could maintain pressure and eliminate the need for viscoelastic in some of these procedures. As a final inspection tool alone its utility is obvious.

In one embodiment of the invention the fluid projection from the device's tip can be shaped to allow hydro dissection of lens cells safely from the lenticular capsule with less volume of fluid required. This can be controlled via a foot pedal or from the hand-piece itself. An example of such an embodiment of the tool is shown in FIG. 15 where various buttons, levers and valves are shown integrated in to the tool, to control the flow rate and other parameters that are described herein. The flow rate may be adjusted based on the requirements of the task and at the discretion of the surgeon. The flow may be pulsed or continuous according to the requirements of the task and at the discretion of the surgeon. Accordingly, the frequency and intensity of the pulse can be set by the surgeon as well. For the purpose of example alone, the frequency of pulsation may be a low rate of a pulse per second or a fast rate of 10 or greater pulses per second. Furthermore, the fluid flow rate and pulse rate can vary independently.

By varying the flow- and pulse-rate the device will adapt to the experience and the tissue differences encountered. The pulsation rate may be controlled by different methods. The pump can stutter in its pump movement, a valve can occlude either flow line course or in the hand-piece itself. Such methods are well understood by one knowledgeable of the arts of fluid flow and the above examples are not limiting.

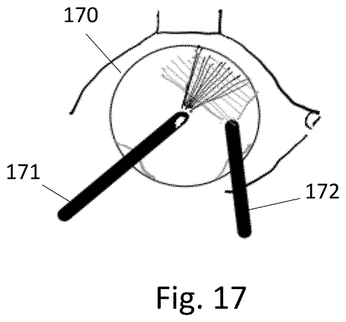

FIG. 17 shows another embodiment of the invention that demonstrates the use of two devices simultaneously. FIG. 17 schematically illustrates combined camera and irrigation probe 171 working together in parallel with a light probe 172 within a human eye 170. Typically one to two incisions (normally called the ‘primary’ and ‘secondary’ incisions) of less than 3 mm are made in the eye during surgery. The device described herein is fitted such that it seals the incision and enables pressure to be retained in the eye during surgery. The two incisions enable the use of two separate devices to be used simultaneously during surgery. For the purposes of example alone, the two devices can be a combination of a number of different features. The first tool can incorporate a camera and irrigation tool inserted in one incision and the second tool can incorporate an illumination probe. An expert in eye surgery will see the utility in such an approach and be able to devise other combinations of features for both tools. For example light illumination (of any wavelength), lasers, irrigation, scraping elements, cameras and other, can be integrated in to either tool in any combination. The unique miniaturized features of the present invention enable the combination of such features to allow the necessary flexibility for an expert in eye surgery to operate optimally.

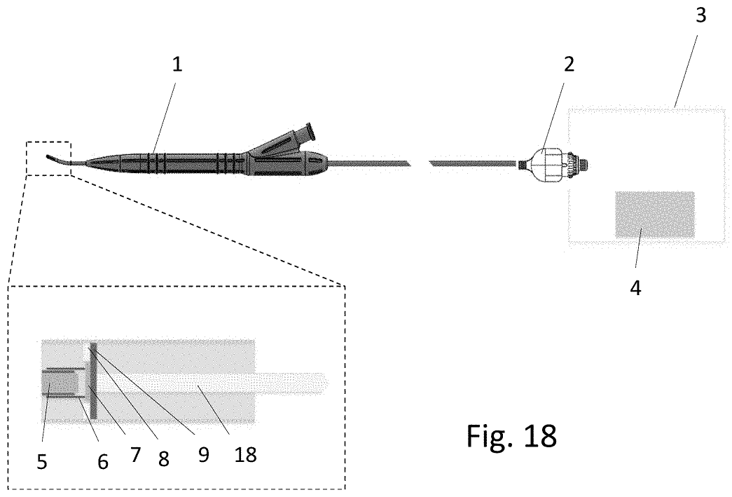

FIG. 18 shows an embodiment of the invention that demonstrates the miniaturization capability of the medical ophthalmic device as well as the different components/units. A handpiece 1 is connected via suitable connector 2 to an endoscopy unit 3 and video controller 4. The endoscopy unit 3 includes several features, but is not limited to the following: white light balance and light intensity controllers, capturing photo-stills and/or live video recording. Furthermore, irrigation regulation and control can be provided through this unit or may be included in a separate unit. Said endoscopy unit is compatible with all electrical connections such as HDMI, DVI, composite, S-Video and USB, but is not limited by this list alone. In this embodiment, the probe consists of various components integrated together at the distal end of the probe unit. In this embodiment, an optic barrel 5 is built of two or more lenses and can contain any number of filters and or/coatings to control any aspect of illumination (e.g. intensity, frequency/wavelength, temporal light intermittence, waveform, etc.) according to a particular need. A sensor housing 6 and the optical barrel 5 can be integrated together or separated, according to the optical requirements of a particular procedure. A sensor 7 can be wafer-based i.e., camera, sensor and optical barrel are integrated/assembled as one piece, or using more conventional methods wherein the optical components are separated from the sensor itself and the focussing lens can be adjusted by changing the distance between their surfaces. One or more LEDs 8 are integrated in to the distal end of the probe device on a Printed Circuit Board (PCB) 9. The light source can also be provided by optical fibres from an external light source unit to the distal end of the probe. The LEDs can be placed where its surface and the distal surface of the optical barrel are flush to further save volumetric space in aiding miniaturization. The LEDs 8 can also be placed on the same PCB as the sensor 6. Furthermore, the camera can also operate without any illumination; such an option may be important in order to further miniaturize the device for high resolution precision surgery. In the option without the illumination features described herein, the probe can operate with a camera at a much smaller scale due to the space saved and subsequent miniaturization. Alternately a larger chip could be used to give higher resolution from the same incision requirements. A cable 18 connects all features throughout the device, from the distal end of the probe, where many of the functional components are located, to the handpiece and external measuring units. A flexible design PCB can be implemented to replace the cable. The PCB can be angled at 90 degrees and, in another option, two PCBs can be used. The cable 18 connects to the video controller 4 in the endoscopy unit 3. Furthermore, a miniature video-controller can be placed on the handpiece itself and integrated into the device.

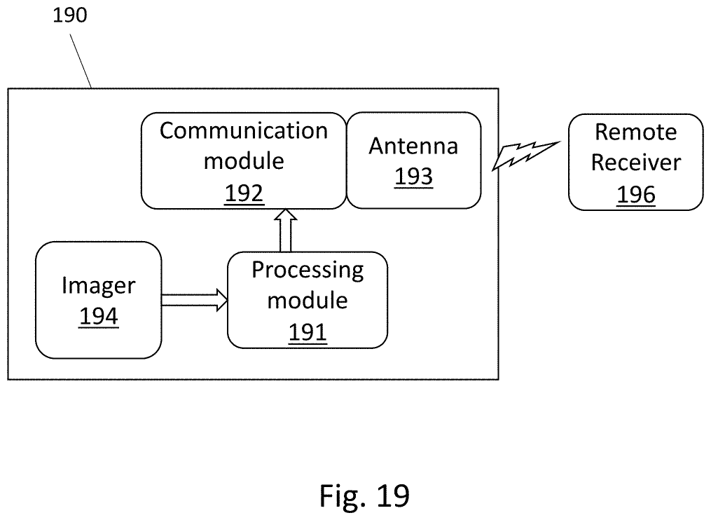

FIG. 19 schematically illustrates a block diagram of the electrical component of a miniature precision medical device 190, according to an embodiment of the invention. Device 190 comprises an imager 194 for converting an optical image viewed by the endoscope into digital image data, a processing module 191 for processing the digital image data, a communication module 192 configured for transmitting the processed digital image data to a remote receiver 196 in a wireless or wired manner, and at least one antenna 193 for directing wireless signals relative to the digital image data to the remote receiver.

As will be appreciated by a skilled person, the processing module may comprise at least one or more processors and a memory.

The tip may be of a size so as to allow insertion into the side port incision or the primary incision. It may be used with the irrigation/aspiration unit in bi-manual mode as a separate and uniquely suited device from the current flow tip used with this tool. The flow-rate affects the velocity of the effluent by creating pressure by injection of fluid via the tip. Therefore the flow rate may vary by the tip opening width and the size of the slit opening. The flow-rate may be between 0 to 100 ml per minute based on aperture size. The width may be 0.5 to 2.5 mm and the opening may be 0.05 mm to 0.5 mm or larger for high flow. The tip opening may be adjustable. The tips may be designed to allow for broader or narrower focus of the flow as well as intensity.

The device may include a curved or straight section behind the tip to allow for gentle motion against the taught capsule for mechanically removing cells or loosening cells to then be swept clear by the tip.

1. A miniature precision medical device comprising an endoscope with at least one camera, wherein at least one sensor of the at least one camera is distally located at a tip of a shaft of said endoscope, characterized with that one or more unaccommodated areas within said shaft, next to said at least one sensor is utilized for accommodating at least one accessory.

2. The device of claim 1, wherein the at least one camera is adapted to provide high resolution images of at least 30K pixels.

3. The device of claim 1, wherein the tip is configured to be inserted through an incision made around the cornea during cataract surgery and minimal invasive glaucoma surgeries (MIGS).

4. The device of claim 1, wherein the tip is configured to be inserted through the trocars used in vitrectomy surgery.

5. The device of claim 1, wherein the diameter of said device is adapted to enable minimally invasive surgeries that employ surgical techniques meant to limit the size of incisions needed of less than 3 mm and so lessen wound healing time, associated pain or risk of infection.

6. The device of claim 5, wherein the diameter of the tip is less than 1.8 mm.

7. The device of claim 1, wherein one or more illumination sources are incorporated in to said device via at least one of the unaccommodated area.

8. The device of claim 7, wherein the illumination source is located distally at the tip or proximally in a hand-piece of the device, or at the connector, or at a video controller.

9. The device of claim 8, wherein the distal illumination source is one or more LEDs.

10. The device of claim 8, wherein light reaches the distal end of the endoscope via optic fibres and/or light guides.

11. The device of claim 1, wherein a phaco-emulsifier tool is integrated in to said device.

12. The device of claim 1, further comprising a tool for removing particles of lenses under direct vision.

13. The device of claim 12, wherein the tool for removing particles of lenses under direct vision is an irrigation tool, an aspiration tool, a camera or combination thereof.

14. The device of claim 13, wherein the irrigation and aspiration are integrated together in concentric circles, wherein one in the inner circle and one in the outer circle, or they are located adjacently.

15. The device of claim 13, wherein the irrigation tool having irrigation port that is shaped to control pressure or direction by fluidics passing through the tip.

16. The device of claim 1, wherein the camera is integrated in to an olive tip irrigation cannula for allowing scarping the lens of the eye.

17. The device of claim 1, wherein the camera is attached to forceps and scissor tools.

18. The device of claim 1, wherein the camera is a video camera.

19. The device of claim 1, wherein the camera is attached to a cannula.

20. The device of claim 1, wherein a laser device is incorporated in to said multi-purpose eye surgery device.

21. The device of claim 1, wherein an external illumination source is adapted to provide illumination in accordance with the orientation of the device/camera.

22. The device of claim 1, wherein a tube or the tip of the endoscope is made of a transparent material, thus it can be used as light guiding generated by an illumination source.

23. The device of claim 7, wherein the wavelength of the light the can be varied.

24. The device of claim 1, wherein the endoscope comprises an imager for converting an optical image viewed by the endoscope into digital image data.

25. The device of claim 24, further comprising a processor for processing the digital image data.

26. The device of claim 25, further comprising a communication module configured for transmitting the processed digital image data to a remote receiver in a wireless or wired manner.

27. The device of claim 26, wherein the communication module comprises one or more antennas for directing wireless signals relative to said digital image data to the remote receiver.

28. The device of claim 1, further comprising a body cavity or an insertion socket through which the endoscope is detachably attachable to the device.

29. The device of claim 25, wherein the processer is part of a control module that is detachably attachable to the device.

30. The device of claim 1, wherein the tip is detachably attachable to a hand-piece of the device.

31. The device of claim 1, further comprising a hand-piece that has a straight tip designed for the back of the eye surgeries.

32. The device of claim 1, further comprising a hand-piece that has a curved tip designed for the front of the eye surgeries.

33. The device of claim 1, a hand-piece of said device has a bendable and articulated tip.
